(19) United States
(12) Patent Application Publication
(10) Pub. No.: US 2016/0209404 A1
(43) Pub. Date: Jul. 21, 2016

(54) PESTICIDE EFFICACY KIT

(71) Applicant: JAB Distributors, LLC, Wheeling, IL (US)

(72) Inventors: Brian Hirsch, Kildeer, IL (US); Warrick Bell, Northbrook, IL (US); Michael F. Potter, Lexington, KY (US); Kenneth F. Haynes, Lexington, KY (US); Jennifer R. Gordon, Racine, WI (US)

(21) Appl. No.: 14/997,679

(22) Filed: Jan. 18, 2016

Related U.S. Application Data
(60) Provisional application No. 62/105,324, filed on Jan. 20, 2015.

(51) Int. Cl. G01N 33/50

(52) U.S. Cl. CPC G01N 33/5085

(57) ABSTRACT

A kit for testing the effectiveness of pesticides. The kit including a plurality of dishes that can be stackably connected to each other, a lid that can be connected to at least one of the plurality of dishes, a substrate that is fitted to be placed in one of the plurality of dishes, and a closeable container that has a compartment for the dishes and a compartment for the substrate.

### RELATED APPLICATIONS

This application makes reference and claims priority to U.S. Provisional Patent Application No. 62/105,324, filed on Jan. 20, 2015, titled “Pesticide Efficacy Kit.” U.S. Provisional Patent Application No. 62/105,324 is hereby incorporated by reference in its entirety.

### FIELD OF EMBODIMENTS OF THE INVENTION

Embodiments of the present invention generally relate to a kit for testing the efficacy of pesticides.

### BACKGROUND

Bed bugs, commonly known as cimex lectularis, are a type of insect that commonly hides within mattresses, box springs, bedding, floors, and furniture. Such bed bugs are found in homes, cruise ships, motels, hostels or boarding houses where itinerant travelers find overnight lodging. Bed bugs feed off of the blood of humans sleeping on the mattresses that harbor these insects. Typically, a bed bug will crawl out of the mattress during the night, bite the sleeping victim, and then return to the safe confines of the mattress.

To treat the problem of bed bugs, pest control professionals visit an infestation site and apply chemical pesticides/insecticides at the site to kill the bed bugs. Such efforts to exterminate the bed bug population at a site typically require multiple visits and treatments.

Different kinds of pesticides may provide different results when it comes to killing bed bugs. Furthermore, some bed bug strains or populations may have developed resistances to certain kinds of pesticides. In that regard, bed bugs are highly adept at becoming resistant to insecticides, and genetic changes in bed bugs that provide resistance to pesticides can occur swiftly. Therefore, insecticide resistance in bed bugs is widespread and becoming a global predicament. In the case of DDT, for instance, some bed bug populations were already becoming resistant within the first few years of use. Bed bugs have genetic mechanisms to resist more modern insecticides as well.

Moreover, no two bed bug infestations are identical in their susceptibility or resistance to insecticides. There can be tremendous variation among the bed bug populations of different infestations, with certain chemical compounds performing well against some populations and poorly on others that have developed a resistance to the particular compounds.

In addition, different formulations of the same insecticide compound (e.g., liquid, dust, spray, etc.) can also vary in their effectiveness against bed bugs. For example, a dust formulation may work better than a liquid formulation on a particular bed bug population. Furthermore, the type of surface to which a compound is applied can affect how effective the compound is. For example, the effectiveness of a compound may vary depending on whether it is applied to fabric or wood.

Many insecticides are marketed as effective against bed bugs. However, for many of these products, efficacy testing is limited and, even when efficacy data are available, the data may not reflect resistance levels of particular bed bug populations encountered in the field. That is because bed bug products are often evaluated using long-maintained laboratory strains of bed bug populations that become more susceptible to pesticides over time.

### SUMMARY

Certain embodiments of the present invention include a kit for testing the effectiveness of pesticides. The kit includes a plurality of dishes that can be stackably connected to each other, a lid that can be connected to at least one of the plurality of dishes, a substrate that is fitted to be placed in and remove from one of the plurality of dishes, and a closeable container that has a compartment for the dishes and a compartment for the substrate.

The kit can further include any of a brush, a magnifying lens, a grease pencil, and a cooling pack. The substrate can be made of one of the following: Masonite, canvas, and woven fabric. The plurality of dishes can be at least four dishes, and each dish can be threadably connected to any of the other dishes. The dishes can be transparent. The plurality of dishes can include a first dish and a second dish and the first dish can be configured to carry the substrate and be threadably connected to the bottom of the second dish to seal the substrate in the first dish. Each dish can include a circular wall that includes threads at a top end on the outside of the wall and threads at a bottom end on the inside of the wall. The substrate can include a rigid portion attached to the back thereof and a flexible tab extending therefrom.

Certain embodiments of the present invention include a kit for testing the effectiveness of pesticides. The kit includes a plurality of dishes that are threadably connectable to and disconnectable from each other, wherein when the dishes are connected to each other, they are stacked on top of each other. The kit further includes a lid that can be connected to at least one of the plurality of dishes, and a substrate that is fitted to be placed in one of the plurality of dishes. The substrate has a tab extending therefrom. The kit includes a closeable container that has a compartment for the dishes and a compartment for the substrate.

Certain embodiments of the present invention include a kit for testing the effectiveness of pesticides. The kit includes a plurality of dishes that are threadably connectable to and disconnectable from each other, wherein each dish includes a circular wall that includes threads at a top end on the outside of the wall and threads at a bottom end on the inside of the wall. The kit includes a lid that can be connected to at least one of the plurality of dishes and a substrate that is fitted to be placed in one of the plurality of dishes. The substrate has a tab extending therefrom. The kit includes a closeable container that has a compartment for the dishes and a compartment for the substrate.

### DESCRIPTION OF THE DRAWINGS

FIG. 1 is a front view of a pesticide kit according to an embodiment of the present invention.

FIG. 2 is an isometric view of the kit of FIG. 1 opened.

FIG. 3 is an isometric view of evaluation dishes used in the kit of FIG. 1.

FIG. 4 is an isometric view of evaluation dishes and substrates used in the kit of FIG. 1.

FIG. 5 is a front view of a tower of evaluation dishes according to an embodiment of the present invention.

FIG. 6 is an isometric view of evaluation dishes and substrates used in the kit of FIG. 1.

FIG. 7 is an isometric view of evaluation dishes and substrates used in the kit of FIG. 1.

FIG. 8 is a front isometric view of a tower of evaluation dishes and substrates used in the kit of FIG. 1.

FIG. 9 is an isometric view of a substrate according to an embodiment of the present invention.

Before the embodiments of the invention are explained in detail, it is to be understood that the invention is not limited in its application to the details of construction and the arrangement of the components set forth in the following description or illustrated in the drawings. The invention is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION OF EMBODIMENTS OF THE INVENTION

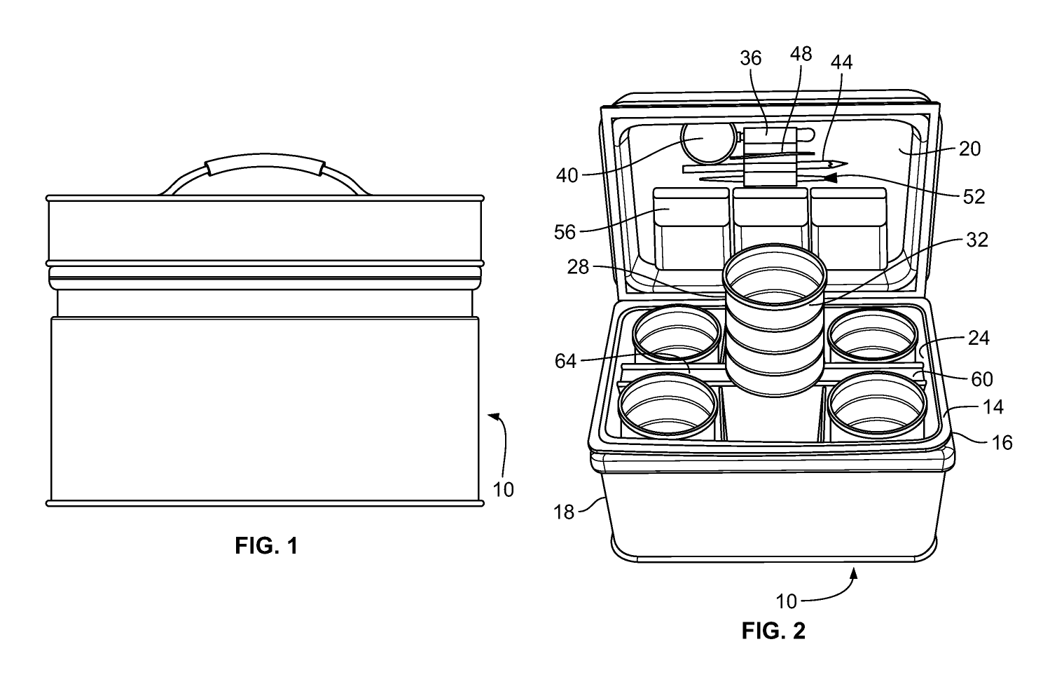

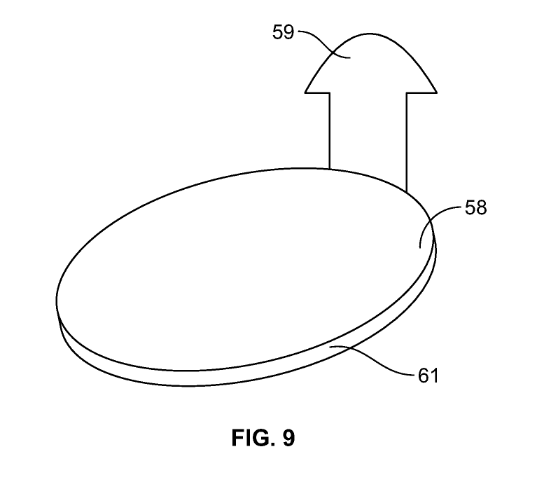

FIGS. 1 and 2 illustrate a pesticide testing kit 10 according to an embodiment of the present invention. The kit 10 includes a cushioned and insulated container or bag 14 that can be opened and closed with a zipper 16. The bag 14 includes a large bottom section 18 and a lid section 20. The bottom section 18 of the bag 14 has compartments 24 to carry towers 28 of modular evaluation dishes 32. The bag 14 can carry six towers 28. Alternatively, the bag 14 can carry another number of 28 towers. The inside of the lid 20 of the bag 14 has fabric loops 36 that hold a magnifying glass 40, a grease pencil 44, specimen collection forceps 48, and a dust application brush 52. The lid 20 of the bag 14 also includes pouches or compartments 56 with cover flaps that hold circular substrates 58 (FIG. 9) for use in the evaluation dishes 32. The cover flaps can be closed over the pouches 48 by use of a hook and loop fastener such as Velcro. Furthermore, the bag 14 can include a compartment or space 60 between the compartments 24 for the towers 28 that can hold an ice or cooling pack 64 or other cooling device.

In general, the modular stackable evaluation dishes 32 are used to hold and transport bed bugs collected at an infestation site. The interchangeable substrates 58 (FIG. 9) are treated with pesticide and placed in the evaluation dishes 32 with the bugs to evaluate the efficacy of the pesticides on the bugs.

The kit 10 can be used by a pest control professional at the time of an initial inspection of a bed bug infestation site. Referring to FIG. 9, the first step is to select substrates 58 from the pouches 56 of the bag 14 to use in the testing. Insecticides tend to work better on some surfaces than others. Surfaces made of materials that readily absorb the insecticide may not be as effective as surfaces on which the insecticide collects and remains and where it can be encountered by the bugs. Bed bugs tend to congregate on fabric, wood, and paper more than metals and plastic. The bag 14 contains substrates 58 made of different materials to simulate use of a pesticide on different materials at an infestation site. For example, the bag 14 can include a substrate made of non-woven fabric, such as polypropylene, to simulate mattress and box spring or bedding fabric, furniture fabric or canvas to simulate upholstery material, and Masonite or particle board to simulate wood materials such as floors, baseboards, or furniture. Thus, the woven and canvas fabric substrates represent surfaces that are porous or absorbent, while the Masonite substrate represents surfaces that are less absorbent. Cardboard or some other rigid structure 61 may be adhered to the backs of the fabric and canvas substrates to provide some additional rigidity to the substrates. When evaluating more than one insecticide product, the user may start with a single kind of substrate. Subsequent evaluations of the products can then be made with alternative substrates of different materials. Alternatively, single product could be tested on several different kinds of substrates to see how the product works on different surface.

The user can treat the substrates 58 with candidate insecticides either before arriving at the site or upon arriving at the site. To do so, the user places some substrates 58 (such as, e.g., a furniture fabric substrate, a Masonite substrate, and/or a bedding fabric substrate) onto sheets of newspaper or paper towels and lightly treats one or both sides of each substrate 58 with the candidate insecticide. Treating both sides of the substrates 58 will help prevent bed bugs from avoiding exposure to the insecticide by crawling underneath the substrates 58 in the evaluation dish 32. Different forms of an insecticide can be applied to the substrates 58. For example, liquid insecticides can be diluted in accordance with instructions on the label and applied with a hand-held sprayer, plant mister, etc., while aerosols can be dispensed directly from the original container. Insecticides formulated as dusts can be applied to the various substrates 58 using the small brush 52 provided with the kit 10. The user will also leave some substrates 58 untreated to use as “control” substrates during the testing of the pesticides.

After treating the substrates 58, the user removes a tower 28 of cylindrical dishes 32 from the bag 14. With reference to FIG. 5, each tower 28 includes four identical modular dishes 32. Alternatively, each tower 28 can have a different number of dishes 32. The dishes 32 can be transparent and, by way of example only, made of plastic. Each dish 32 has an upper receptacle portion 68 and a bottom portion 72. The receptacle portion 68 and bottom portion 72 are separated by a circular base 76. Each dish 32 includes a cylindrical wall 80 that extends along both the upper receptacle portion 68 and the bottom portion 72.

The wall 80 along the upper receptacle portion 68 includes threads 84 on the exterior thereof, and the wall 80 along the bottom portion 72 includes threads 88 on the interior thereof that correspond to the threads 84 on the upper receptacle portion 68 of another dish 32. Therefore, any of the dishes 32 in a tower 28 can be threadably connected to another of the dishes 32 in the tower 28, and, in this way, the dishes 32 in a tower 28 are modular and interchangeable with each other. Screwing one dish 32 on top of another dish 32 seals the inside of the lower dish 32 from the outside environment, and four dishes 32 can be screwed together to form a tower 28. The top dish 32 can be closed with a circular lid 92 that includes threads 96 that match the threads 84 on the exterior of the upper receptacle portion 68 of the dish 32. In this way, bed bugs can be sealed in each of four dishes 32 in a tower 28 along with a substrate 58.

The portion of the wall 80 of each dish 32 that extends below the base 76 to define the bottom portion 72 of the dish 32 serves to position the base 76 of the dish 32 above the surface on which the dish 32 is rested. In this way, the bottom of the base 76 of a dish 32 does not come into contact with liquids and other substances on whatever surface the dish 32 may be placed when detached from the tower 28. Therefore, a dish 32 that has been sitting on a counter is less likely to contaminate another dish 32 that is subsequently screwed to the bottom of it.

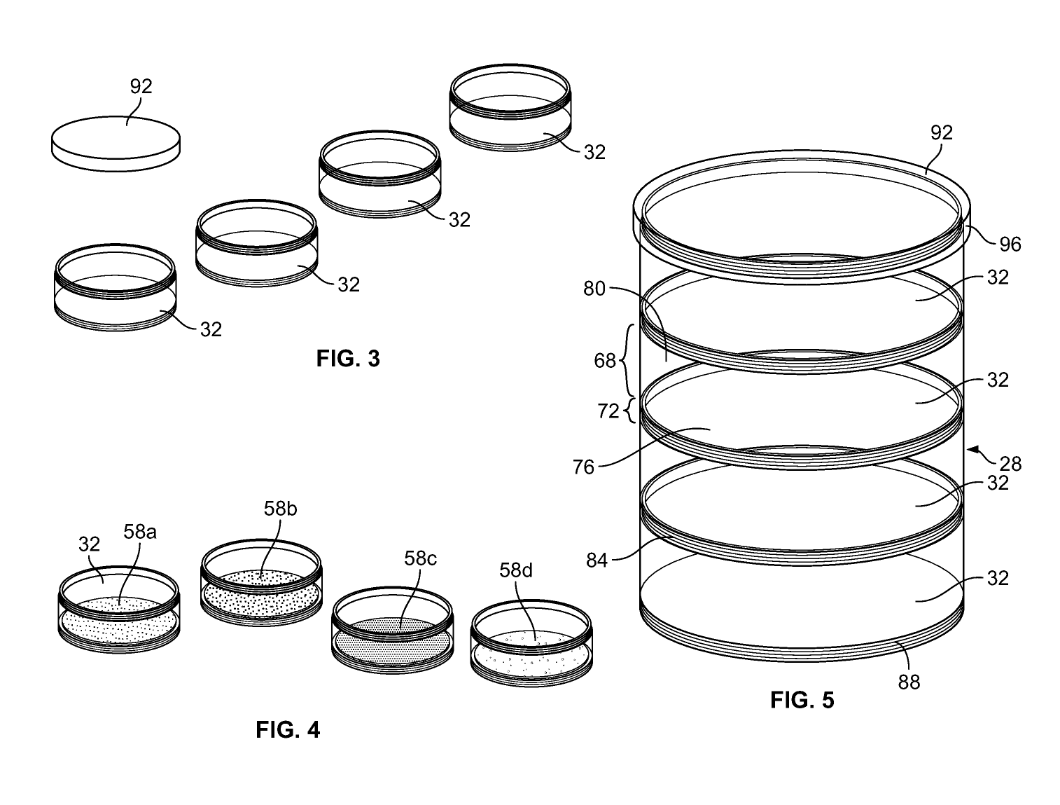

As shown in FIGS. 3 and 4, upon removing a tower 28 of dishes 32 from the bag 14, the user unscrews the lid 92 of the topmost dish 32. The user can then insert a substrate 58 into that dish 32. By way of example, the user can insert an untreated or control substrate 58a into the bottom of the dish 32. With reference to FIG. 9, each substrate 58 can have a tab 59 attached thereto to facilitate insertion into and removal from a dish 32. Prior to insertion of a substrate 58 into a dish 32, the tab 59 can be folded down against the top of substrate 58. When the substrate 58 has been inserted into a dish 32, the tab 59 can be folded to extend upward along the inner side of the dish wall 80 in order to aid in later removal of the substrate 58 from the dish 32. The tab 59 may be made of plastic or another thin, flexible material.

Returning to FIG. 4, once the untreated substrate 58a has been inserted into the dish 32, the user can then use the grease pencil 44 or some other writing device from the bag 14 to label the dish 32 to indicate that no pesticide was applied to the substrate 58a in the dish 32.

The other substrates 58b-d can be treated with insecticide. When the treated substrates 58b-d are dry, the user can proceed with unscrewing and opening the other dishes 32 in the tower stack 28 and inserting each of the substrates 58b-d that has been treated with pesticide into a dish 32. By way of example only, substrate 58b is made of Masonite, substrate 58c is made of woven fabric, and substrate 58d is made of canvas. Pressing each treated substrate 58b-d flush along the base of a dish 32 reduces the chance of a bug crawling beneath the treated substrate 58b-d. The user then uses the grease pencil 44 to label each dish 32 that includes a treated substrate 58b-d with information related to which pesticide in what form was used with the substrate 58 placed in the dish 32. The grease pencil 44 can also be used to label the dish 32 to indicate the location, date, and time of the testing. Extra treated substrates 58 can be stored for future use in appropriately labeled and sealed plastic bags in a cool place.

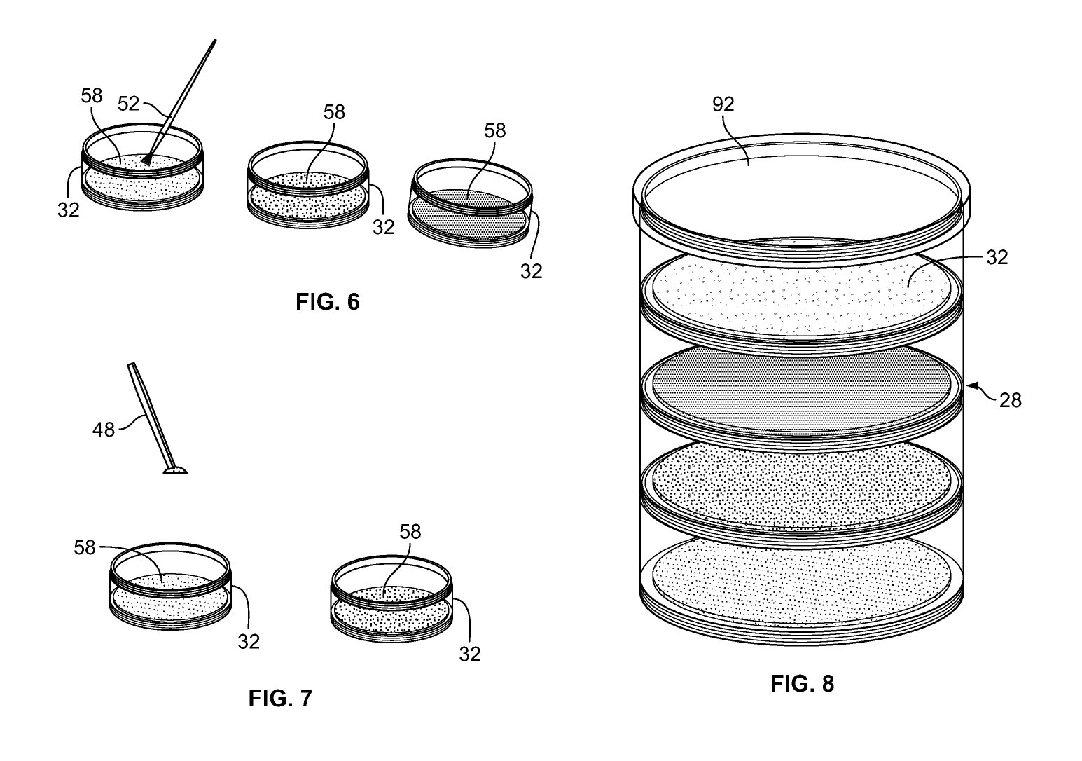

Alternatively, with reference to FIG. 6, instead of being treated with pesticides before being placed in the dishes 32, the substrates 58 can be placed in the dishes 32 and treated with a pesticide while in the dish 32. The user can flip the substrate 58 over in the dish 32 to treat both sides of the substrate 58.

With reference to FIG. 7, once the substrates 58 (untreated 58a and treated 58b-d) have been placed in the separated dishes 32, the user can use the forceps 48 to collect bed bugs from the infestation site and place live bugs in each of the dishes 32 on top of a substrate 58. The forceps 48 have soft tips to help prevent killing the bed bugs during collection. For more reliable test results, the user can put around the same number of live bugs into each evaluation dish 32. The user may add bed bugs to the topmost dish 32 containing the untreated control substrate 58a first in order to avoid contamination of that dish 32 with any pesticide applied to the other substrates 58b-d which the forceps 48 may contact. The untreated dish 32 can also serve as a bulk collection reservoir for collected bugs before the user places the bugs in the other dishes 32 that contain the treated substrates 58b-d.

Putting multiple bugs in each dish 32 will help improve the reliability of the test results. For example, a user may put a minimum of 4-5 bed bugs in each dish 32. In addition, for larger infestations, after filling the dishes 32 with bugs, a user may want to collect larger quantities of additional bugs in a plastic bag to take back to the office for additional testing.

With reference to FIG. 8, once bugs have been placed in each of the dishes 32, the dishes 32 are screwed together and re-assembled into a tower 28, and the lid 92 is screwed on the top dish 32. The tower 28 is then placed back in the bag 14, and the user can close the bag 14 and go on to another site.

Bed bugs can die from exposure to heat. If bugs collected from an infestation site die from heat in the bag 14 during transportation in a hot vehicle from the site back to the office or lab, the user will not necessarily know if the bugs died from heat or due to the effectiveness of the pesticide on the substrate 58 to which the bugs were exposed in a dish 32. The cooling pack 64 (FIG. 2) and insulation of the bag 14 helps to prevent collected bugs from dying of heat in the closed bag 14 during transportation and thus helps prevent the skewing of testing results. Consequently, a user may take the bag 14 on several jobs during the course of a hot day and make collections of bugs at each site before taking the bag 14 and the collected bugs back to the office or lab without having to worry about the collected bugs dying from heat exposure during the course of the day.

Once the user has returned back to the office from collections at infestation sites, the user can remove the towers 28 from the bag 14 and store the towers 28 on a shelf or desktop for subsequent evaluation. A user can examine the dishes 32 in a tower 28 on at least a daily basis to record the number of bugs that die in each dish 32 due to exposure to a particular pesticide on a particular substrate 58 in the dish 32. The user can use the magnifying lens 40 to examine the bugs. Mortality can be confirmed by opening the dish 32 and gently touching the hind end of the bug with the supplied forceps 48 or a pencil. If the bed bug is able to crawl forward, it can be considered alive. Survival of bugs in the dish 32 carrying the untreated control substrate 58a provides evidence that the mortality of bugs in other dishes 32 is due to the insecticide instead of other factors.

The onset and progression of mortality of bed bugs depends on the resistance level of each bed bug population. While susceptible bed bug population strains may succumb within a few hours of exposure to the insecticides, highly resistant ones may persist much longer with many of the individuals appearing to be unaffected. For example, two different strains of bed bugs could react differently to the same kind of insecticide. For most insecticides, users of the kit 10 should be able to make meaningful determinations about their effectiveness on bed bugs within 1-2 days or possibly a week. Some products, however, may be slower acting, requiring somewhat longer evaluation periods.

The user can thus use the towers 28 of dishes 32 to evaluate how fast particular pesticides in particular forms applied to particular substrate materials kill a bed bug from a particular infestation site, if at all. Based on that evaluation, the user can then make a determination as to the most effective insecticide product to use at that particular site upon returning to the site for treatment. For example, examination of a tower 28 (or multiple towers 28) of bugs taken from a particular site may show that the bug population at that site is especially susceptible to a specific powder insecticide on a fabric surface and a specific liquid insecticide on a wooden surface. Thus, the kit 10 can help provide specific “point-of-care” information early on with respect to the infestation site that is to be treated and save a user from having to make repeated visits and experiment with different types of pesticides if a particular pesticide does not work effectively after the first treatment.

After evaluations are completed, the dishes 32 of the tower 28 can be separated from each other, emptied, thoroughly cleaned, and then re-connected to re-form a tower 28 that can be transported in the bag 14 for use at a new site.

The kit 10 can be used in other situations besides testing products for a specific bed bug infestation. For example, the kit 10 can be used to evaluate different pesticide or insecticide products of interest before purchasing bulk quantities of the products. Moreover, the kit can be used to test chemicals on other insects or pests besides bed bugs. By way of example only, the dishes 32 can be used to collect ants, stink bugs, and various roaches and beetles.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A kit for testing the effectiveness of pesticides, comprising:
a plurality of dishes that can be stackably connected to each other to form a stacked dish tower;
a lid that can be connected to at least one of the plurality of dishes;
a plurality of substrates that are fitted to be placed in and removed from the plurality of dishes, wherein the substrates are configured to be treated with pesticide before being placed in the dishes;
a closeable container that has a compartment for the dish tower and a compartment for the plurality of substrates.

2. The kit of claim 1, further including a brush.

3. The kit of claim 1, further including a magnifying lens.

4. The kit of claim 1, further including a grease pencil.

5. The kit of claim 1, wherein the container is insulated and further includes a cooling pack and forceps.

6. The kit of claim 1, wherein the substrate is made of one of the following: Masonite, canvas, and woven fabric.

7. The kit of claim 1, wherein the plurality of dishes is at least four dishes, and each dish can be threadably connected to any of the other dishes.

8. The kit of claim 1, wherein the dishes are transparent.

9. The kit of claim 1, wherein the compartment for the dish tower is located in a base portion of the container and the compartment for the substrates is located in a lid of the container.

10. The kit of claim 1, wherein the plurality of dishes includes a first dish and a second dish and the first dish is configured to carry the substrate and be threadably connected to the bottom of the second dish to seal the substrate in the first dish.

11. The kit of claim 1, wherein each dish includes a circular wall that includes threads at a top end on the outside of the wall and threads at a bottom end on the inside of the wall.

12. The kit of claim 1, wherein the substrate includes a rigid portion attached to the back thereof and a flexible tab extending therefrom.

13. A kit for testing the effectiveness of pesticides, comprising:
a plurality of dishes that are threadably connectable to and disconnectable from each other, wherein when the dishes are connected to each other, they are stacked on top of each other;
a lid that can be connected to at least one of the plurality of dishes;
a substrate that is fitted to be placed in one of the plurality of dishes, wherein the substrate generally has the same form before and after it is placed in one of the plurality of dishes and is configured to be treated with pesticide before being placed in the dish and has a tab extending therefrom, and
a closeable container that has a compartment for the dishes and a compartment for the substrate.

14. The kit of claim 13, further including a brush, a magnifying lens, forceps, and a writing utensil.

15. The kit of claim 13, wherein the container is insulated and further includes a cooling pack.

16. The kit of claim 13, wherein the substrate is made of one of the following: Masonite, canvas, and woven fabric.

17. The kit of claim 13, wherein the plurality of dishes includes a first dish and a second dish and the first dish is configured to carry the substrate and be threadably connected to the bottom of the second dish to seal the substrate in the first dish.

18. The kit of claim 13, wherein each dish includes a circular wall that includes threads at a top end on the outside of the wall and threads at a bottom end on the inside of the wall.

19. A kit for testing the effectiveness of pesticides, comprising:
a plurality of dishes that are threadably connectable to and disconnectable from each other, wherein each dish includes a circular wall that includes threads at a top end on the outside of the wall and threads at a bottom end on the inside of the wall;
a lid that can be connected to at least one of the plurality of dishes;
a substrate that is fitted to be placed in one of the plurality of dishes, wherein the substrate generally has the same form before and after it is placed in one of the plurality of dishes and is configured to be treated with pesticide before being placed in the dish and has a tab extending therefrom, and
a closeable container that has a compartment for the dishes and a compartment for the substrate.

20. The kit of claim 19, wherein the plurality of dishes includes a first dish and a second dish and the first dish is configured to carry the substrate and be threadably connected to the bottom of the second dish to seal the substrate in the first dish.
